(19) United States
(12) Patent Application Publication
(10) Pub. No.: US 2018/0369282 A1
(43) Pub. Date: Dec. 27, 2018

(54) GENE-MODIFIED LYMPHOCYTES EXPRESSING CHIMERIC ANTIGEN RECEPTOR IN WHICH PRODUCTION OF INFLAMMATORY CYTOKINES IS INHIBITED

(71) Applicants: National University Corporation Nagoya University, Nagoya-shi (JP); Shinshu University, Matsumoto City (JP)

(72) Inventors: Nobuhiro Nishio, Nagoya-shi (JP); Yoshiyuki Takahashi, Nagoya-shi (JP); Yozo Nakazawa, Matsumoto City (JP); Kazuyuki Matsuda, Matsumoto City (JP)

(21) Appl. No.: 15/632,604

(22) Filed: Jun. 26, 2017

(51) Int. Cl. A61K 35/17; C12N 15/113; C07K 14/705; C12N 15/85

(52) U.S. Cl. CPC A61K 35/17; C12N 15/1136; C12N 2800/90; C12N 15/85; C12N 2310/14; C07K 14/70503

(57) ABSTRACT

With the aim of improving the treatment result of CAR therapy, intended is to provide an effective means for the cytokine release syndrome, as an alternative to the administration of the anti-IL-6 receptor antibody or the like. Together with the target antigen-specific chimeric antigen receptor gene, a first nucleic acid construct which intracellularly producing an siRNA targeting interleukin-6 gene and/or a second nucleic acid construct which intracellularly producing an siRNA targeting tumor necrosis factor α gene are introduced into the target cell, thus preparing the gene-modified lymphocyte expressing chimeric antigen receptor.

### FIELD OF THE INVENTION

The present invention relates to gene-modified lymphocytes (CAR gene-introduced lymphocytes) expressing a chimeric antigen receptor, and specifically to the method for preparing CAR gene-introduced lymphocytes in which a specific cytokine gene is knocked down, and the use of the cells.

### BACKGROUND OF THE INVENTION

Gene-modified T-cell therapy (CAR-T therapy) and gene-modified NK cell therapy (CAR-NK therapy) using a chimeric antigen receptor (hereinafter may be referred to as “CAR”) find more and more clinical application. A CAR typically has a structure composed of a single chain variable region of an antibody as the extracellular domain, to which linked are a transmembrane region, CD3ξ, and an intracellular domain of a molecule which transmits costimulatory signals. The CAR gene-introduced lymphocytes are activated by binding to the antigen according to specificity of the antibody, and injures the target cells (for example, cancer cells). CAR therapy has advantages such as relatively easy cell preparation, high cytotoxic activity, and sustainable effect, and thus is expected as a new treatment means for refractory subjects and subjects having resistance to conventional therapy. In the actual clinical trials carried out in Europe and the United States, the CAR for the CD19 antigen expressed on the cell surface was gene-introduced into the peripheral blood T-cells collected from patients with chemotherapy-resistant acute lymphoblastic leukemia, cultured, and infused; satisfactory results with a remission rate of 80 to 90% was reported (Grupp S A et al., N Engl J Med, 368(16): 1509-18. 2013; Maude S L et al., N Engl J Med, 371(16): 1507-17.2014; Lee D W et al., Lancet. 2015 Feb. 7; 385(9967): 517-28.). In the United States, CAR therapy has been attracting attention as one of the most promising therapies for refractory cancer.

On the other hand, in the clinical trial of the CAR therapy targeting CD19 antigen for B-cell neoplasms, hypercytokinemia occurs when the CAR gene-introduced T-cells (CAR-T-cells) develops antitumor effect, and serious complications such as acute respiratory distress syndrome, consciousness barrier, and multiorgan failure are found in about 30% patients (Called “cytokine release syndrome”; Maude S L et al., N Engl J Med, 371(16): 1507-17.2014; Lee D W et al., Lancet. 2015 Feb. 7; 385(9967): 517-28; Davila M L Sci Transl Med. 2014; 6: 224ra25.; Xu X J Cancer Lett. 2014 28; 343: 172-8.). Major cytokines which have been reported to cause excessive production are interferon (IFN) γ, tumor necrosis factor (TNF) α, interleukin (IL)-2, IL-6, IL-7, IL-8, IL-10, and IL-12 (Xu X J Cancer Lett. 2014 28; 343: 172-8.). According to the report, adrenocortical steroids, anti-IL-6 receptor antibodies, anti-TNF-α antibodies were administered to cytokine release syndrome caused by CAR therapy, and the administration of anti-IL-6 receptor antibodies were effective.

### SUMMARY OF THE INVENTION

The above-listed measures against cytokine release syndrome (administration of, for example, an adrenocortical steroid agent, an anti-IL-6 receptor antibody, or an anti-TNF-α antibody) showed certain effect on inhibition or relief of cytokine release syndrome. However, these measures are therapeutic intervention after appearance of cytokine release syndrome, and thus are not sufficiently effective at preventing the development of serious complications. In addition, the anti-IL-6 receptor antibody and anti-TNF-α antibody are expensive, and thus impose heavy economical burdens on patients. In addition, since they are antibody molecules, it is concerned that side effects can be caused by unexpected immune reaction. Accordingly, the present invention is intended to provide an effective measure against cytokine release syndrome that replaces the administration of anti-IL-6 receptor antibody or the like, aiming at improvement in treatment recodes of CAR therapy.

In the study for solving the above-described problems, we devised a strategy of knocking down the IL-6 gene by siRNA during preparation of the CAR-T-cells, and verified its effectiveness by using leukemia cells (CD19-positive ALL cells). As a result of this, surprisingly, proliferation of the leukemia cells was completely inhibited, while the production of IL-6, which is a dominant cytokine of the cytokine release syndrome, was not observed. More specifically, production of IL-6 was effectively inhibited, while the intrinsic effect of the CAR-T-cells (cell injury activity) was maintained. This result suggests that the knockdown of the IL-6 gene in the preparation of the CAR-T-cells, or the introduction of the CAR gene allows more efficient and cost-effective inhibition or relief of the cytokine release syndrome than the case using the anti-IL-6 receptor antibody. In the cytokine release syndrome, commonly, excess production of IL-6 is found along with excessive production of the tumor necrosis factor α (TNF-α). In consideration of this fact, the same effect can be expected by knocking down the TNF-α gene as in the case of IL-6. In addition, the effect will be enhanced by knocking down both of the IL-6 gene and TNF-α gene.

The following invention is based on the results and discussions described above.

[1] A method for preparing a gene-modified lymphocyte expressing chimeric antigen receptor, including a step of introducing a target antigen-specific chimeric antigen receptor gene and a first nucleic acid construct which intracellularly produces an siRNA targeting interleukin-6 gene, and/or a second nucleic acid construct which intracellularly produces an siRNA targeting tumor necrosis factor α gene into a target cell.

[2] The preparation method of [1], wherein the introduction of the target antigen-specific chimeric antigen receptor gene, the first nucleic acid construct, and the second nucleic acid construct is carried out by a transposon method.

[3] The preparation method of [2], wherein the transposon method is the piggyBac transposon method.

[4] The preparation method of any one of [1] to [3], wherein the target antigen-specific chimeric antigen receptor gene, the first nucleic acid construct and/or the second expression construct are included in the same vector, and the vector is introduced into the target cell.

[5] The preparation method of any one of [1] to [4], wherein the target cell is T-cell.

[6] A gene-modified lymphocyte obtained by the preparation method of any one of [1] to [5], which expresses the chimeric antigen receptor and intracellularly produces the siRNA targeting interleukin-6 gene and/or the siRNA targeting tumor necrosis factor α gene.

[7] A cell preparation including the gene-modified lymphocyte of [6].

[8] A method for treating cancer including a step of administering the gene-modified lymphocyte of [6] to a cancer patient in a therapeutically effective amount.

[9] A vector including a chimeric antigen receptor expression cassette containing a target antigen-specific chimeric antigen receptor gene, and an siRNA expression cassette containing a first nucleic acid construct intracellularly producing an siRNA targeting interleukin-6 gene, and/or a second nucleic acid construct intracellularly producing an siRNA targeting tumor necrosis factor α gene.

[10] The vector according to [9], which includes a structure wherein the chimeric antigen receptor expression cassette and the siRNA expression cassette are sandwiched between a pair of transposon inverted repeat sequences.

[11] A kit for preparing a gene-modified lymphocyte expressing chimeric antigen receptor, including the vector according to [10], and a transposase expression vector.

[12] A kit for preparing a gene-modified lymphocyte expressing chimeric antigen receptor, including:

a vector including a chimeric antigen receptor expression cassette containing the target antigen-specific chimeric antigen receptor gene, and

a vector including an siRNA expression cassette containing a first nucleic acid construct intracellularly producing an siRNA targeting interleukin-6 gene, and/or a second nucleic acid construct intracellularly producing an siRNA targeting tumor necrosis factor α gene.

[13] The preparation kit of [12], wherein the chimeric antigen receptor expression cassette has a structure sandwiched by a pair of transposon inverted repeat sequences,

the siRNA expression cassette has a structure sandwiched between a pair of transposon inverted repeat sequences, and

the kit further includes a transposase expression vector.

[14] A kit for preparing a gene-modified lymphocyte expressing chimeric antigen receptor, including:

a vector including a first chimeric antigen receptor expression cassette containing the target antigen-specific chimeric antigen receptor gene,

a vector including a first siRNA expression cassette containing a first nucleic acid construct intracellularly producing an siRNA targeting interleukin-6 gene, and

a vector including a second siRNA expression cassette containing a second nucleic acid construct intracellularly producing an siRNA targeting tumor necrosis factor α gene.

[15] The preparation kit of [14], wherein the chimeric antigen receptor expression cassette has a structure sandwiched between a pair of transposon inverted repeat sequences,

the first siRNA expression cassette has a structure sandwiched between a pair of transposon inverted repeat sequences,

the second siRNA expression cassette has a structure sandwiched between a pair of transposon inverted repeat sequences, and

the kit further includes a transposase expression vector.

[16] The preparation kit of any one of [11], [13], and [15], wherein the transposase is piggyBac transposase.

### BRIEF DESCRIPTION OF THE DRAWINGS

These and other objectives and technical advantages of the present invention will be readily apparent from the following description of the preferred exemplary embodiments of the invention in conjunction with the accompanying drawings, in which:

FIG. 1 shows the structure of the pIRII-CAR.CD19-IL6KD vector (6680 bps). In addition to the leader sequence (SEQ ID NO: 24) and the sequences coding the CD19 CAR (light chain variable region (SEQ ID NO: 25), heavy chain variable region (SEQ ID NO: 26), Fc region (SEQ ID NO: 27), transmembrane region and intracellular domain of CD28 (SEQ ID NO: 28), and CD3ξ (SEQ ID NO: 29)), the sequence coding the shRNA targeting IL-6 gene are included. SEQ ID NO: 11 shows the sequence of the full length of the vector.

FIG. 2 shows the DNA fragment (SEQ ID NO: 15) for the RNAi targeting IL-6 gene. The sequence coding the U6 promoter (underlined; SEQ ID NO: 16) and shRNA (double underlined; SEQ ID NO: 17) are included.

FIG. 3 shows the structure of the pIRII-CAR.CD19-TNFaKD vector (6683 bps). In addition to the leader sequence (SEQ ID NO: 24) and the sequences coding the CD19 CAR (light chain variable region (SEQ ID NO: 25), heavy chain variable region (SEQ ID NO: 26), Fc region (SEQ ID NO: 27), transmembrane region and intracellular domain of CD28 (SEQ ID NO: 28), and CD3ξ (SEQ ID NO: 29)), the sequence coding the shRNA targeting TNF-α gene are included. The sequences of the full length of the vector are shown in SEQ ID NO: 12 (Example No. 1), SEQ ID NO: 13 (Example No. 2), and SEQ ID NO: 14 (Example No. 3).

FIG. 4 shows the examples of the DNA fragment (Example No. 1 is SEQ ID NO: 18, Example No. 2 is SEQ ID NO: 19, and Example No. 3 is SEQ ID NO: 20) for RNAi targeting TNF-α gene. The sequences coding the U6 promoter (underlined; SEQ ID NO: 16) and shRNAs (double underlined; Example No. 1 is SEQ ID NO: 21, Example No. 2 is SEQ ID NO: 22, and Example No. 3 is SEQ ID NO: 23) are included.

FIG. 5 shows the result of the co-culture experiment of the CD19 CAR/IL6KD-T-cells and acute lymphoblastic leukemia (ALL) cell line. ALL: acute lymphoblastic leukemia, IL-6: interleukin-6, KD: knockdown

### DETAILED DESCRIPTION OF THE INVENTION

1. Preparation of Gene-Modified Lymphocyte Expressing Chimeric Antigen Receptor

The present invention relates to a method for preparing the gene-modified lymphocyte expressing chimeric antigen receptor (CAR gene-introduced lymphocytes). The CAR gene-introduced lymphocytes obtained by the preparation method of the present invention can be used for CAR therapy. The preparation method of the present invention includes the following step: introducing into target cells the target antigen-specific chimeric antigen receptor (CAR) gene and a first nucleic acid construct which intracellularly produces an siRNA targeting interleukin-6 (hereinafter referred to as “IL-6”) gene (hereinafter referred to as “IL-6 siRNA”), and/or a second nucleic acid construct which intracellularly produces an siRNA targeting tumor necrosis factor α (hereinafter referred to as “TNF-α”) gene (hereinafter referred to as “TNF-α siRNA”). This step provides the cells expressing the target antigen-specific CAR gene and the IL-6 siRNA (when the first nucleic acid construct is introduced), the cells expressing the target antigen-specific CAR gene and TNF-α siRNA (when the second nucleic acid construct is introduced), or the cells expressing the target antigen-specific CAR gene, IL-6 siRNA, and TNF-α siRNA (when both of the first and second nucleic acid constructs are introduced). Unless otherwise specified, the cells (for example, T-cells) herein are human cells.

The CAR gene codes the chimeric antigen receptor (CAR) recognizing a specific target antigen. The CAR is a structural body including an extracellular domain specific to the target, a transmembrane domain, and an intracellular signal domain for the effector function of immunocytes. These domains are explained below.

(a) Extracellular Domain

The extracellular domain specifically binds to the target. For example, the extracellular domain contains the scFv fragment of the anti-target monoclonal antibody. Examples of the monoclonal antibody used herein include rodent antibodies (e.g., mouse, rat, and rabbit antibodies), human antibodies, and humanized antibodies. The humanized monoclonal antibody is prepared by making the structure of the monoclonal antibody of any animal species (for example, mice or rats) analogous to the structure of the human antibody, and includes the human type chimera antibody, which is prepared by substituting only the constant region of an antibody with that of the human antibody, and the human type CDR-grafted antibody, which is prepared by substituting the parts excluding the CDR (complementary determining region) in the constant and variable regions with those of the human antibody (P. T. Johons et al., Nature 321, 522 (1986)). For the purpose of increasing the antigen binding activity of human type CDR-grafted antibodies, already developed are the improvement techniques for the method for choosing a human antibody framework (FR) having high homology for mouse antibodies, the method for preparing humanized antibodies having high homology, and the method for transplanting a mouse CDR in a human antibody, followed by substitution of amino acids in the FR region (e.g., U.S. Pat. No. 5,585,089, U. S. Pat. No. 5,693,761, U.S. Pat. No. 5,693,762, U.S. Pat. No. 6,180,370, European Patent Application No. 451216, European Patent Application No. 682040, and Japanese Patent No. 2828340), which can be used for the preparation of humanized antibodies.

The scFv fragment is a structural body wherein the light chain variable region (VL) and heavy chain variable region (VH) of immunoglobulin are linked through a linker, and retains binding ability for the antigen. The linker may be, for example, a peptide linker. The peptide linker is composed of a peptide made by linear linking of amino acids. Typical examples of the peptide linker are the linkers composed of glycine and serine (GGS and GS linkers). The amino acids composing the GGS and GS linkers, glycine and serine, are small in their sizes, and thus hardly form higher-order structures. The length of the linker is not particularly limited. For example, a linker having 5 to 25 amino acid residues may be used. The number of the amino acid residue composing the linker is preferably from 8 to 25, and more preferably from 15 to 20.

The target used herein is typically an antigen which shows specific expression in tumor cells. The “specific expression” means significant or remarkable expression in comparison with the cells other than tumor, and will not intend to confine to those showing no expression in the cells other than tumor. Examples of the target antigen include the CD19 antigen, CD20 antigen, GD2 antigen, CD22 antigen, CD30 antigen, CD33 antigen, CD44 variant 7/8 antigen, CD123 antigen, CEA antigen, Her2/neu antigen, MUC1 antigen, MUC4 antigen, MUC6 antigen, IL-13 receptor-alpha 2, immunoglobulin light chain, PSMA antigen, VEGF receptor 2, mesothelin antigen, EGFR vIII, EphA2 antigen, and IGFR.

(b) Transmembrane Domain

The transmembrane domain intervenes between the extracellular domain and intracellular signal domain. Examples of the transmembrane domain used herein include CD28, CD3ε, CD8α, CD3, CD4, and 4-1BB. Alternatively, a transmembrane domain composed of an artificially constructed polypeptide may be used.

(c) Intracellular Signal Domain

The intracellular signal domain transmits the signals necessary for exertion of the effector function of immunocytes. More specifically, when the extracellular domain binds with the target antigen, an intracellular signal domain capable of transmitting the signals necessary for activation of immunocytes are used. The intracellular signal domain includes the domain for transmitting the signals through the TCR complex (for convenience, referred to as “the first domain”), and the domain for transmitting the costimulatory signals (for convenience, referred to as “the second domain”). As the first domain, CD3ξ or other intracellular domains such as FcεRIγ may be used. The use of CD3ξ is preferred. As the second domain, the intracellular domain of a costimulatory molecule is used. Examples of the costimulatory molecule include CD28, 4-1BB (CD137), CD2, CD4, CD5, CD134, OX-40, and ICOS. The use of the intracellular domain of CD28 or 4-1BB is preferred.

The linking form of the first and second domains is not particularly limited, and preferably the second domain is disposed on the transmembrane domain side, because it is known that co-stimulation was strongly transmitted when CD3ξ was linked distally in a past case. The same or different kinds of plural intracellular domains may be linked in tandem to form the first domain. The same holds true for the second domain.

The first and second domains may be directly linked, or a linker may intervene between them. The linker may be, for example, a peptide linker. The peptide linker is composed of peptides which are linear chains of amino acids. The structure and characteristics of the peptide linker are as described above. However, the linker used herein may be composed solely of glycine. The length of the linker is not particularly limited. For example, a linker composed of 2 to 15 amino acid residues may be used.

(d) Other Elements

A leader sequence (signal peptide) is used to promote CAR secretion. For example, the leader sequence of the GM-CSF receptor may be used. In addition, the structure is preferably composed of an extracellular domain and a transmembrane domain linked together through a spacer domain. More specifically, the CAR according to a preferred embodiment contains a spacer domain between the extracellular domain and transmembrane domain. The spacer domain is used for promoting linking between the CAR and target antigen. For example, the Fc fragment of a human IgG (for example, human IgG1 or human IgG4) may be used as the spacer domain. Alternatively, a part of the extracellular domain of CD28 or a part of the extracellular domain of CD8α may be used as the spacer domain. A spacer domain may be placed between the transmembrane domain and intracellular signal domain.

There are some reports on the experiments and clinical studies using CARs (for example, Rossig C, et al. Mol Ther 10: 5-18, 2004; Dotti G, et al. Hum Gene Ther 20: 1229-1239, 2009; Ngo M C, et al. Hum Mol Genet 20 (R1): R93-99, 2011; Ahmed N, et al. Mol Ther 17: 1779-1787, 2009; Pule M A, et al. Nat Med 14: 1264-1270, 2008; Louis C U, et al. Blood 118: 6050-6056, 2011; Kochenderfer J N, et al. Blood 116: 4099-4102, 2010; Kochenderfer J N, et al. Blood 119: 2709-2720, 2012; Porter D L, et al. N Engl J Med 365: 725-733, 2011; Kalos M, et al. Sci Transl Med 3: 95ra73,2011; Brentjens R J, et al. Blood 118: 4817-4828, 2011; and Brentjens R J, et al. Sci Transl Med 5: 177 ra38, 2013), and the CARs in the present invention may be constructed with reference to these reports.

The first nucleic acid construct intracellularly producing an IL-6 siRNA and the second nucleic acid construct intracellularly producing a TNF-α siRNA are used for expression inhibition by so-called RNAi (RNA interference). In other words, the introduction of the first nucleic acid construct and/or the second expression construct into the target cell allows inhibition of the expression of the target gene (IL-6, TNF-α) by RNAi in the target cell. For convenience of explanation, the first and second nucleic acid constructs may be generically referred to as “siRNA construct”.

RNAi is the process of sequence-specific gene inhibition after transcription which can be caused in eukaryotic cells. In the RNAi for mammal cells, a double-strand RNA (siRNA) having a short sequence corresponding to the sequence of the target mRNA is used. Usually, the siRNA has 21 to 23 base pairs. Mammal cells are known to have two pathways (a sequence-specific pathway and a sequence-nonspecific pathway) which are influenced by the double-strand RNA (dsRNA). In the sequence-specific pathway, a relatively long dsRNA is divided into short-interfering RNAs (more specifically, siRNAs). On the other hand, the sequence-nonspecific pathway is considered as induced by any dsRNA irrespective of the sequence, as long as it is not shorter than a certain length. In this pathway, the dsRNA activates two enzymes, or PKR, which is activated to phosphorylate the translation initiation factor eIF2 for entirely stopping protein synthesis, and 2′,5′-oligoadenylic acid synthase, which participates in the synthesis of the RNAase L activation molecules. In order to minimize the progress of the nonspecific pathway, the use of a double-strand RNA (siRNA) shorter than 30 base pairs is preferred (see Hunter et al. (1975) J Biol Chem 250: 409-17; Manche et al. (1992) Mol Cell Biol 12: 5239-48; Minks et al. (1979) J Biol Chem 254: 10180-3; and Elbashir et al. (2001) Nature 411: 494-8).

In order to produce a target-specific RNAi, an siRNA, which is composed of a sense RNA which is homology with a part of the mRNA sequence of the target gene, and its complementary antisense RNA, is expressed intracellularly. The first and second expression constructs achieve this expression.

The siRNA targeting the specific gene (target gene) is usually a double-strand RNA made by hybridization of a sense RNA composed of a sequence homologous to the continuous region in the mRNA sequence of the gene, and an antisense RNA composed of the complementary sequence. The length of the “continuous region” is usually 15 to 30 bases, preferably 18 to 23 bases, and more preferably 19 to 21 bases.

It is known that a double-strand RNA having overhangs of servral bases at the ends exerts high RNAi effect. Accordingly, in the present invention, the use of the siRNA having such structure is preferred. The length of the base forming each of the overhangs is not particularly limited, and preferably 2 bases (for example, TT and UU).

The siRNA may be designed by an ordinary method. Designining of the siRNA usually uses a sequence characteristic to the target sequence (continuous sequence). In addition, programs and algorithms for choosing appropriate target sequences have been developed.

The “nucleic acid construct intracellularly producing an siRNA” means nucleic acid molecules whose introduction into cells causes a desired siRNA (the siRNA causing RNAi for the target gene) by an intracellular process. Typically, a nucleic acid construct is constructed so as to express the shRNA to be converted to siRNA by the subsequent process, and included in an appropriate vector. In this manner, an siRNA vector referred to as stem loop type or short hairpin type (a vector into which a sequence coding shRNA is inserted), or an siRNA vector referred to as tandem type (a vector expressing a sense RNA and an antisense RNA separately) is obtained. These vectors can be made by those skilled in the art according to common procedure (for example, refere to Brummelkamp T R et al. (2002) Science 296: 550-553; Lee N S et al. (2001) Nature Biotechnology 19: 500-505; Miyagishi M & Taira K (2002) Nature Biotechnology 19: 497-500; Paddison P J et al. (2002) Proc. Natl. Acad. Sci. USA 99: 1443-1448; Paul C P et al. (2002) Nature Biotechnology 19: 505-508; Sui G et al. (2002) Proc Natl Acad Sci USA 99(8): 5515-5520; and Paddison P J et al. (2002) Genes Dev.16: 948-958). The shRNA has a structure (hairpin structure) wherein a sense RNA and an antisense RNA are linked through a loop structure, the loop structure is intracellularly cleaved to form double-strand siRNA, and exert the RNAi effect. The length of the loop structure is not particularly limited, but is usually from 3 to 23 bases.

The genes whose expression is inhibited in the present invention are the IL-6 gene and/or TNF-α gene. The sequence of the IL-6 gene registered in a public database is shown in SEQ ID NO: 1 (Accession No. NM _000600, Definition: Homo sapiens interleukin-6 (IL6)), and the sequence of the TNF-α gene also registered is shown in SEQ ID NO: 2 (Accession No. NM _000594, Definition: Homo sapiens tumor necrosis factor (TNF), mRNA). An example of the sequence of IL-6 siRNA (sense strand) is shown in SEQ ID NO: 3, and the sequence of the shRNA corresponding to the IL-6 siRNA is shown in SEQ ID NO: 4. In the same manner, examples of the sequence of TNF-α siRNA (sense strand) are shown in SEQ ID NOs: 5 to 7, and the sequences of the shRNA corresponding to these TNF-α siRNAs are shown in SEQ ID NOs: 8 to 10.

The shRNA itself may be used as the siRNA construct. In this case, the vector including the CAR gene and the siRNA construct are introduced into the target cell.

In a preferred embodiment of the present invention, an expression cassette containing the CAR gene (CAR expression cassette), and an expression cassette containing the first nucleic acid construct and/or the second nucleic acid construct (siRNA expression cassette) are included in the same vector. In this framework, only one vector (referred to as “CAR-siRNA vector”) is introduced into the target cell, which simplifies the gene introduction operation necessary for the preparation method of the present invention.

Alternatively, a vector including a CAR expression cassette (CAR vector) and a vector including an siRNA expression cassette (siRNA vector) are prepared, and these vectors are introduced to the target cell. The order of introduction is not particularly limited, but it is preferred that the introduction of the CAR vector be preceded. When this order is used, it is preferable to introduce the siRNA vector after selecting, concentrating and/or purifying the target cell into which the CAR vector has been appropriately introduced. This improves the making efficiency and purity of the desired CAR gene-introduced lymphocytes (more specifically, lymphocytes which express CAR, and further express IL-6 siRNA and/or TNF-α siRNA). At present, various RNAi vectors are available. The vector including an siRNA expression cassette may be constructed using such known vectors. For example, an insert DNA coding the desired RNA (for example, shRNA) is prepered, then inserted into the cloning site of the vector, and used as an RNAi expression vector. The origin and structure of the vector are not limited, as long as it has a function of intracellularly expressing an siRNA exerting RNAi action on the target gene. As the siRNA vector, a vector including the IL-6 siRNA expression cassette and/or a vector including the TNF-α siRNA expression cassette, or, a vector including the IL-6 siRNA expression cassette and TNF-α expression cassette is used.

Examples of the promoter used in the CAR expression cassette include CMV-IE (cytomegalovirus early gene-derived promoter), SV40ori, retrovirus LTP, SRα, EF1α, and β actin promoter. The promoter is operably linked to the CAR gene. “The promoter is operably linked to the CAR gene.” has the same meaning with “the CAR gene is disposed under control of the promoter”, and usually, the CAR gene is linked to the 3′ terminal side of the promoter directly or via other sequence. A poly-A additional signal sequence is disposed downstream of the CAR gene. Transcription is terminated by the use of the poly-A additional signal sequence. The poly-A additional signal sequence may be, for example, the poly-A additional sequence of SV40, or the poly-A additional sequence of a bovine-derived growth hormone gene.

Examples of the promoter used in the siRNA expression cassette include U6 promoter, H1 promoter, and tRNA promoter. These promoters are RNA polymerase III promoters, and expected to achieve a high expression efficiency.

The above-described vectors (CAR-siRNA vector, CAR vector, and siRNA vector) may include, for example, a gene for detection (for example, a reporter gene, cell or tissue-specific gene, or selectable marker gene), an enhancer sequence, and a WRPE sequence. The gene for detection is used for the judgement of success/failure or efficiency of the introduction the expression cassette, detection of CAR expression or judgement of the expression efficiency, and selection and collection of the cells having expressed the CAR gene. On the other hand, the enhancer sequence is used for improving the expression efficiency. Examples of the gene for detection include the neo gene imparting resistance against neomycin, the npt gene (Herrera Estrella, EMBO J.2 (1983), 987-995) and npt II gene (Messing & Vierra.Gene 1 9: 259-268(1982)) imparting resistance against kanamycin, the hph gene imparting resistance against hygromycin (Blochinger & Diggl mann, Mol Cell Bio 4: 2929-2931), and the dhfr gene imparting resistance against Methotrexate (Bourouis et al., EMBO J.2(7)) (the aforementioned are marker genes); genes of fluorescence proteins such as the luciferase gene (Giacomin, P1.Sci.116 (1996), 59 to 72; Scikantha, J. Bact. 178 (1996), 121), the β-glucuronidase (GUS) gene, GFP (Gerdes, FEBS Lett.389 (1996), 44-47), and their variants (EGFP and d2EGFP) (the aforementioned are reporter genes); and the epidarmal growth factor receptor (EGFR) gene deficient in the intracellular domain. The gene for detection is linked to the CAR gene through, for example, a bicistronic control sequence (for example, internal ribosome entry site (IRES)) and a sequence coding a self cleavage peptide. Examples of the self cleavage peptide include, but not limited to, the 2A peptide (T2A) derived from Thosea asigna virus. Known examples of the self cleavage peptide include the 2A peptide (F2A) defived from the Foo-and-mouse disease virus (FMDV), the 2A peptide (E2A) defived from equine rhinitis A virus (ERAV), and the 2A peptide (P2A) defived from porcine teschovirus (PTV-1).

The introduction of the CAR gene, and the first and second nucleic acid constructs may use various gene introduction methods. The gene introduction methods are roughly divided into the methods using a viral vector and non-viral vectors. The former cleverly uses the phenomonon of the infection of a virus to a cell, and provides a high gene introduction efficiency. As the viral vectors, for example, retrovirus vector, lentivirus vector, adenovirus vector, adeno-associated virus vector, herpesvirus vector, and Sendai virus vector have been developed. Among them, the retrovirus vector, lentivirus vector, and adeno-associated virus vector are expected to achieve stable and long-term expression, because the target genes included in these vectors are integrated in the host chromosomes. These viral vectors can be prepared according to known methods, or using a commercially available kit. Examples of the non-viral vector include plasmid vector, liposome vector, positively charged liposome vector (Feigner, P. L., Gadek, T. R., Holm, M. et al., Proc. Natl. Acad. Sci., 84: 7413-7417, 1987), YAC vector, and BAC vector.

The gene introduction is preferably carried out by a transposon method. The transposon method is one of the non-viral gene introduction methods. Transposon is the generic name of short gene sequences causing a gene transposition conserved during the process of evolution. A pair of a gene enzyme (transposase) and its specific recognition sequence causes gene transposition. The transposon method may be, for example, the piggyBac transposon method. The piggyBac transposon method uses the transposon isolated from insects (Fraser M J et al., Insect Mol Biol. 1996 May; 5(2): 141-51.; Wilson M H et al., Mol Ther. 2007 January; 15(1): 139-45.), and allows highly efficient integration into mammal chromosomes. The piggyBac transposon method is actually used for the introduction of the CAR gene (for example, see Nakazawa Y, et al., J Immunother 32: 826-836, 2009; Nakazawa Y et al., J Immunother 6: 3-10, 2013). The transposon method applicable to the present invention is not limited to that using piggyBac, and may use a method using transposon, for example, Sleeping Beauty (Ivics Z, Hackett P B, Plasterk R H, Izsvak Z (1997) Cell 91: 501-510.), Frog Prince (Miskey C, Izsvak Z, Plasterk R H, Ivics Z (2003) Nucleic Acids Res 31: 6873-6881.), Toll (Koga A, Inagaki H, Bessho Y, Hori H. Mol Gen Genet. 1995 Dec. 10; 249 (4): 400-5.; Koga A, Shimada A, Kuroki T, Hori H, Kusumi J, Kyono-Hamaguchi Y, Hamaguchi S. J Hum Genet. 2007; 52(7): 628-35.Epub 2007 Jun. 7.), To12 (Koga A, Hori H, Sakaizumi M (2002) Mar Biotechnol 4: 6-11.; Johnson Ha mL et M R, Yergeau D A, Kuliyev E, Takeda M, Taira M, Kawakami K, Mead P E (2006) Genesis 44: 438-445.; Choo B G, Kondrichin I, Parinov S, Emelyanov A, Go W, Toh W C, and Korzh V (2006) BMC Dev Biol 6: 5.).

The introduction operation by the transposon method may be carried out by an ordinary method with reference to past literatures (for example, for the piggyBac transposon method, see Nakazawa Y, et al., J Immunother 32: 826-836, 2009, Nakazawa Y et al., J Immunother 6: 3-10, 2013, Saha S, Nakazawa Y, Huye L E, Doherty J E, Galvan D L, Rooney C M, Wilson M H. J Vis Exp. 2012 Nov. 5; (69): e4235, Saito S, Nakazawa Y, Sueki A, et al. Anti-leukemic potency of piggyBac-mediated CD19-specific T cells against refractory Philadelphia chromosome-positive acute lymphoblastic leukemia. Cytotherapy. 2014; 16: 1257-69.).

In a preferred embodiment of the present invention, the piggyBac transposon method is used. Typically, in the piggyBac transposon method, a vector including the gene coding piggyBac transposase (transposase plasmid) and a vector having a structure wherein the desired nucleic acid construct (CAR expression cassette and/or siRNA expression cassette) is sandwiched between piggyBac inverted repeat sequences (transposon plasmid) are prepered, and these vectors are introduced (transfected) to the target cell. The transfection may use various methods such as electroporation, nucleofection, lipofection, or calcium phosphate method.

Examples of a target cell (the cell into which the CAR gene and the first and/or second nucleic acid constructs are introduced) include CD4-positive CD8-negative T-cells, CD4-negative CD8-positive T-cells, T-cells prepared from iPS cells, αβ-T-cells, γδ-T-cells, NK cells, and NKT cells. Various cell populations may be used, as long as they contain the above-described lymphocytes or precursor cells. PBMCs (peripheral blood mononuclear cells) collected from the peripheral blood is one of the preferred target cells. More specifically, in a preferred embodiment, gene introduction operation is carried out on the PBMCs. The PBMCs may be prepared by an ordinary method. The method for preparing the PBMCs may refer to, for example, Saha S, Nakazawa Y, Huye L E, Doherty J E, Galvan D L, Rooney C M, Wilson M H. J Vis Exp. 2012 Nov. 5; (69): e4235.

The CAR gene-introduced lymphocytes obtained by the above steps are typically subjected to activation treatment. For example, the CAR gene-introduced lymphocytes are activated by stimulation with an anti-CD3 antibody and an anti-CD28 antibody. This treatment also usually promotes survival and proliferation of the CAR gene-introduced lymphocytes. For example, stimulation by the anti-CD3 antibody and anti-CD28 antibody can be applied by culturing in a culture vessel (for example, culture dish) coated with the anti-CD3 antibody and anti-CD28 antibody on the culture surface for 1 to 20 days, preferably 3 to 14 days, and more preferably 5 to 10 days. The anti-CD3 antibody (for example, CD3pure antibody provided by Miltenyi Biotec) and the anti-CD28 antibody (for example, CD28pure antibody provided by Miltenyi Biotec) are readily and commercially available. Magnetic beads (for example, Dynabeads T-Activator CD3/CD28 provided by VERITAS) coated with the anti-CD3 antibody and anti-CD28 antibody may be used to apply the stimulation. The anti-CD3 antibody is preferably “OKT3” clone. In order to promote recovery from injury/disturbance by gene introduction operation, the activation treatment is preferably carried out about 8 to 48 hours (preferably 16 to 24 hours) after the gene introduction operation, rather than immediately after the gene introduction operation.

In order to improve the survival rate/proliferation rate of the cells, it is preferred to use a culture solution containing a T-cell growth factor in the activation treatment. The T-cell growth factor is preferably IL-15. Preferably, a culture solution containing IL-15 and IL-7 is used. The concentration of IL-15 is, for example, from 1 ng/mL to 20 ng/mL, and preferably from 5 ng/mL to 10 ng/mL. The concentration of IL-7 is, for example, from 1 ng/mL to 20 ng/mL, and preferably from 5 ng/mL to 10 ng/mL. The T-cell growth factor such as IL-15 or IL-7 may be prepared according to a common procedure. Alternatively, a commercial product may be used. Although the use of animal T-cell growth factors other than human ones will not be excluded, the T-cell growth factor used herein is usually derived from human (may be a recombinant). The growth factors such as human IL-15 and human IL-7 are readily available (for example, provided by Miltenyi Biotec, R&D systems).

A medium containing blood serum (for example, human blood serum or fetal bovine serum) may be used, but the use of a serum-free medium allows the preparation of cells having advantages of high safety in clinical application, and safe advantages of a high level of safety and little difference in the culture efficiency among blood serum lots. Specific example of the serum-free medium for lymphocytes include TexMACS™ (Miltenyi Biotec) and AIM V (registered trademark) (Thermo Fisher Scientific). When a blood serum is used, the blood serum is preferably an autologous serum, or a blood serum collected from a patient to receive administration of CAR gene-introduced lymphocytes obtained by the preparation method of the present invention. The basal culture medium is the one suitable for culture of lymphocytes, and a specific example is the above-listed TexMACS™, AIM V (registered trademark). Other culture conditions may be common ones, as long as they are suitable for the survival and proliferation of lymphocytes. For example, the lyphocytes are cultred in a CO₂ incubator adjusted at 37° C. (CO₂ concentration: 5%).

After activating treatment, the cells are collected. The collecting operation may follow an ordinary method. For example, the cells are collected by pipetting or centrifugation. In one preferred embodiment, before the collecting operation, the cells after activating treatment is cultured in the presence of a T-cell growth factor. This step allows efficient expanded culture, and increases the cell survival rate. The T-cell growth factor used herein may be, for example, IL-15 or IL-7. In the same manner as in the activating treatment, the cells may be cultured in a medium containing IL-15 and IL-7. The culture period is for example from 1 to 21 days, preferably from 5 to 18 days, and more preferably from 10 to 14 days. If the culture period is too short, the number of cells will not sufficiently increase, and if the culture period is too long, cell activity (survival ability) may decrease, and the cell may cause exhaustion/fatigue or the like. The cells may be subcultured during the culture. During the culture, the medium is replaced as necessary. For example, about ⅓ to ⅔ the culture solution is replaced with a new medium once in three days.

2. CAR Gene-Introduced Lymphocytes and and Uses Thereof

The second aspect of the present invention relates to the gene-modified lymphocyte expressing chemeric antigen receptors obtained in the preparation method of the present invention (hereinafter referred to as “CAR gene-introduced lymphocytes of the present invention”) and uses thereof. The CAR gene-introduced lymphocytes of the present invention can be used for treatment, prevention, or improvement of various diseases (hereinafter referred to as “target diseases”) to which the CAR therapy is likely effective. Representative examples of the target disease include, but not limited to, cancer. Examples of the target disease include various B-cell lymphoma (follicular lymphoma, diffuse large B-cell lymphoma, mantle cell lymphoma, MALT lymphoma, intravascular B-cell lymphoma, and CD20-positive Hodgkin's lymphoma), myeloproliferative tumor, myelodysplastic/myeloproliferative tumor (CMML, JMML, CML, MDS/MPN-UC), myelodysplastic syndromes, acute myelocytic leukemia, neuroblastoma, brain tumor, Ewing's sarcoma, osteosarcoma, retinoblastoma, small cell lung cancer, melanoma, ovarian cancer, rhabdomyosarcoma, kidney cancer, pancreas cancer, malignant mesothelioma, and prostate cancer. “Treatment” include alleviation (moderation) of symptoms or associated symptoms characteristic to the target diseases, inhibition or retard of deterioration of symptoms. “Prevention” means prevention or retard of development/expression of diseases (disorders) or their symptoms, or decrease of the risk of development/expression. On the other hand, “improvement” means alleviation (moderation), change for the better, amelioration, or healing (containing partial healing).

The CAR gene-introduced lymphocytes of the present invention may be prepered in the form of a cell preparation. The cell preparation of the present invention contains the CAR gene-introduced lymphocytes of the present invention in a therapeutically effective amount. For example, 1×10⁴ to 1×10¹⁰ cells are contained for one administration (one dose). The cell preparation may contain dimethylsulfoxide (DMSO) or serum albumin for the purpose of cell protection, antibiotics for the purpose of preventing bacterial contamination, and various components for (for example, vitamins, cytokine, growth factors, and steroids) for the purpose of cell activation, proliferation, or inductive differentiation.

The administration route of the CAR gene-introduced lymphocytes or cell preparation of the present invention is not particularly limited. For example, they are administered by intravenous injection, intraarterial injection, intraportal injection, intradermal injection, hypodermic injection, intramuscular injection, or intraperitoneal injection. Local administration may be used in place of systemic administration. Examples of the local administration include direct injection into the target tissues, body parts, and organs. The administration schedule may be made according to the sex, age, body weight, and pathology of the subject (patient). A single dose or continuous or periodical multiple doses may be used.

According the previous reports, cytokine release syndrome presents a problem particular in the early stage of treatment (for example, see Non-Patent Literature 3). Therefore, in order to enhance the intrinsic treatment effect of CAR gene-introduced lymphocytes while caring the cytokine release syndrome in the early treatment stage, the CAR gene-introduced lymphocytes of the present invention are used in the early stage of treatment (for example, first dose), and ordinary CAR gene-introduced lymphocytes which will not express IL-6 siRNA nor TNF-α siRNA in the subsequent treatment.

3. Vector and Kit for Preparing CAR Gene-Introduced Lymphocytes

Another aspect of the present invention relate to a vector (CAR gene-introduced lymphocyte preparation vector) and a kit (CAR gene-introduced lymphocyte preparation kit) usable in the preparation method of the present invention. The CAR gene-introduced lymphocyte preparation vector of the present invention includes a CAR expression cassette and an siRNA expression cassette (more specifically, CAR-siRNA vector), and allows the introduction of the two expression cassettes into the target cell using only one vector. The CAR expression cassette includes the CAR gene, a promoter necessary for the expression of the CAR gene (for example, CMV-IE, SV40ori, retrovirus LTP, SRα, EF1α, or β actin promoter). The siRNA expression cassette contains an siRNA construct, more specifically, a nucleic acid construct intracellularly producing IL-6 siRNA (the first nucleic acid construct), and/or a nucleic acid construct intracellularly producing TNF-α siRNA (the second nucleic acid construct), and a promoter necessary for the expression of the siRNA construct (for example, U6 promoter, H1 promoter, or tRNA promoter). The vector of the present invention may include a gene for detection (for example, reporter gene, cell or tissue-specific gene, or selectable marker gene), an enhancer sequence, and a WRPE sequence.

Preferably, the vector of the present invention is constructed as a vector used in the transposon method. In this case, typically, the vector has a structure wherein a CAR expression cassette and an siRNA expression cassette are sandwiched between a pair of transposon inverted repeat sequences (for example, they are disposed in this order: 5′ end transposon inverted repeat sequence, CAR expression cassette, siRNA expression cassette, and 3′ end transposon inverted repeat sequence).

One embodiment of the kit of the present invention is suitable to the method for preparing CAR gene-introduced lymphocytes using the transposon method. The kit contains the above-described CAR-siRNA vector including “CAR expression cassette and siRNA expression cassette” sandwiched between a pair of transposon inverted repeat sequences, and a transposase expression vector. The transposase is selected so as to correspond to the pair of transposon inverted repeat sequences integrated into the CAR-siRNA vector. For example, a combination of a piggyBac inverted repeat sequence and piggyBac transposase is used.

Another embodiment of the kit of the present invention contains vectors which are roughly divided into two kinds, or the CAR vector and siRNA vector. In other words, in this kit, the CAR expression cassette and the siRNA expression cassette are included in different vectors. When this kit is used, for example, the target cell is co-transformed (co-transfected) by the CAR vector and the siRNA vector, or the target cell is transfected by one vector, and then the transformant (vector introduction target cell) is transformed by the other vector. As the siRNA vector, a vector including an IL-6 siRNA expression cassette and/or a vector including a TNF-α siRNA expression cassette, or a vector including an IL-6 siRNA expression cassette and TNF-α expression cassette is used. These vectors must have the above-described structure (see the section 1.). These vectors are constructed so as to be useful for the viral gene introduction method or non-viral introduction method. Preferably, these vectors are constructed so as to be suitable to the gene introduction in the transposon method, which is one of the non-viral introduction methods. More specifically, the CAR vector is constructed so as to have a structure in which the CAR expression cassette is sandwiched between a pair of transposon inverted repeat sequences, and, in the same manner, the siRNA vector is constructed so as to have a structure in which the siRNA expression cassette is sandwiched between a pair of transposon inverted repeat sequences. The kit also includes a transposase expression vector for supplying transposase. The pair of transposon inverted repeat sequences integrated into the CAR vector and siRNA vector are to be subjected to the action of transposase expressed by the transposase expression vector to be combined. More specifically, they are structured in such a manner that the transposon corresponds to the transposon inverted repeat sequences.

The kit of the present invention may include the reagent, instrument, or apparatus used for the gene introduction operation, and the reagent, instrument, or apparatus used for the detection and selection of the transformant. An instruction manual is usually attached to the kit of the present invention.

### EXAMPLES

In order to establish an effective countermeasure to the cytokine release syndrome, which is a problem with the CAR therapy, the following study was carried out.

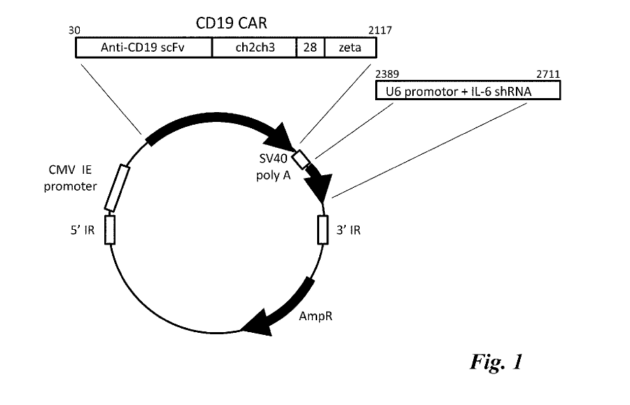

1. Materials and methods

<Preparation of pIRII-CAR.CD19-IL6KD vector (FIG. 1)>

(1) The previously reported piggyBac transposon vector expressing CD19 CAR (pIRII-CAR.CD19, Huye L E, Nakazawa Y, Patel M P, et al. Combining mTor inhibitors with rapamycin-resistant T cells: a two-pronged approach to tumor elimination. Mol Ther. 2011; 19: 2239-48.; Saito S, Nakazawa Y, Sueki A, et al. Anti-leukemic potency of piggyBac-mediated CD19-specific T cells against refractory Philadelphia chromosome-positive acute lymphoblastic leukemia. Cytotherapy. 2014; 16: 1257-69.) was cleaved by both the restrictive enzymes MunI and ClaI.

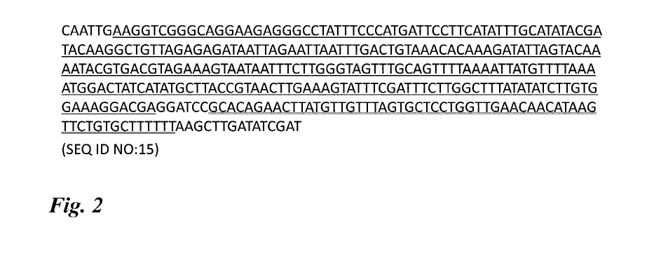

(2) The DNA fragment having the shRNA (IL6KD) sequence inhibiting the expression of the IL-6 gene under control of the U6 promoter, and restriction enzyme recognition sequences (MunI recognition sequence on the 5′ side, and ClaI recognition sequence on the 3′ side) at both sides (FIG. 2, SEQ ID NO: 15; containing U6 promoter (SEQ ID NO: 16) and the sequence coding shRNA (SEQ ID NO: 17)) was cleaved by both MunI and ClaI.

(3) The DNA fragment of 6341 bp obtained in (1) and the DNA fragment of 339 bp obtained in (2) were ligated using the T4 DNA ligase.

(4) The circular DNA plasmid of 6680 bp obtained in (3) was amplified in a large amount using a competent cell.

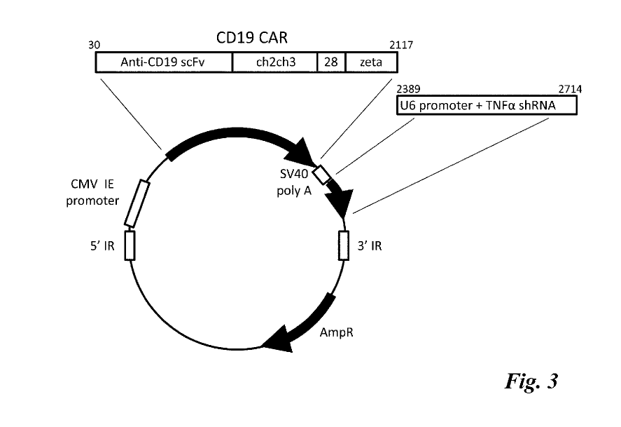

(5) The entire base sequence (SEQ ID NO: 11) was confirmed using a sequencer.

<Preparation of pIRII-CAR.CD19-TNFaKD Vector (FIG. 3)>

(1) The previously reported piggyBac transposon vector expressing CD19 CAR was cleaved by both the restrictive enzymes MunI and ClaI.

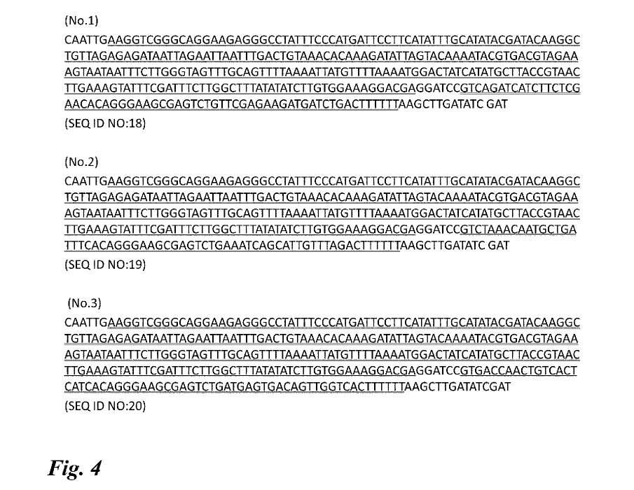

(2) Three kinds of DNA fragments having the shRNA (TNFaKD) sequence inhibiting the expression of the TNF-α gene under control of the U6 promoter, and restriction enzyme recognition sequences (MunI recognition sequence on the 5′ side, and ClaI recognition sequence on the 3′ side) at both sides (FIG. 4, SEQ ID NOs: 18, 19, and 20; each containing U6 promoter (SEQ ID NO: 16) and the sequence coding shRNA (SEQ ID NOs: 21, 22, and 23)) were cleaved by MunI and ClaI.

(3) The DNA fragment of 6341 bp obtained in (1) and the three kinds of DNA fragments of 342 bp obtained in (2) were individually ligated using the T4 DNA ligase.

(4) The circular DNA plasmid of 6683 bp obtained in (3) was amplified in a large amount using a competent cell.

(5) The entire base sequences (SEQ ID NOs: 12 to 14) were confirmed using a sequencer.

<Preparation of CD19 CAR/IL6KD-T-Cells>

(1) Peripheral blood mononuclear cells (PBMCs) were isolated from about 10 mL of peripheral blood using specific gravity centrifuge method.

(2) The pIRII-CAR.CD19-IL6KD vector (5 μg) and pCMV-piggyBac vector (5 μg) were gene-introduced into 1×10⁷ PBMCs by the electroporation method (Program EO-115) using the combination of the 4D-Nucleofector™ apparatus and the P3 Primary Cell 4D-Nucleofector™ X kit (Lonza).

(3) The gene-introduced cells obtained in (2) were allowed to stand in one well of a 24-well culture plate filled with 2 ml of TexMACS™ culture medium (Miltenyi Biotec) containing interleukin (IL)-15 (5 ng/mL, Miltenyi Biotec). After 16 to 24 hours, the gene-introduced cells were transferred together with 2 mL of the culture medium to one well of the 24-well culture plate to which the anti-CD3 antibody (Miltenyi Biotec) and anti-CD28 antibody (Miltenyi Biotec) had been solid-phased. Four days after the gene introduction, the gene-introduced cells were transferred to one well of the non-solid-phased 24 well culture plate. At that time, 1 mL of the TexMACS™ culture medium containing IL-15 was replaced. Seven days after the gene introduction, gene-introduced cells were transferred to the G-Rex10 culture vessel (Wilson Wolf Manufacturing Inc, New Brighton, Minn.) filled with 30 mL of the TexMACS™ culture medium containing IL-15 (5 ng/mL). The cells were collected 14 days after the gene introduction (CD19 CAR/IL6KD-T-cells). Using some of the cells, expression of CD19 CAR protein was confirmed by flow cytometry. As a control group, the conventional CD19 CAR-T-cells, to which the previously reported pIRII-CAR.CD19 vector had been gene-introduced, and T-cells without gene introduction (mock T-cells) were amplified cultured in the same manner.

<Co-Culture Experiment>

1×10⁵ each of mock T-cells, CD19 CAR-T-cells, and CD19 CAR/IL6KD-T-cells were co-cultured one by one of a 48-well culture plate with 5×10⁵ acute lymphoblastic leukemia (ALL) cell line SU/SR at a cell ratio of T-cells:leukemia cells=1:5 in 1 mL portions of 10% fetal bovine serum-containing RPMI1640 culture medium for 5 days. 0.5 mL of the culture supernatant on day 3 of co-culture was collected, and 0.5 mL of the RPMI culture medium containing 10% fetal bovine serum was added. On day 5 after initiation of co-culture, the cells were separately collected from each well, the number of the live cells was counted by trypan blue staining, stained with the anti-CD3-APC antibody and anti-CD19-PE antibody, and then the ratio between the CD3-positive cells (T-cells) and CD19-positive cells (ALL cells) was measured by flow cytometry. In addition, the IL-6 concentration of the culture supernatant collected 3 days after initiation of co-culture was measured by the ELISA method.

2. Results

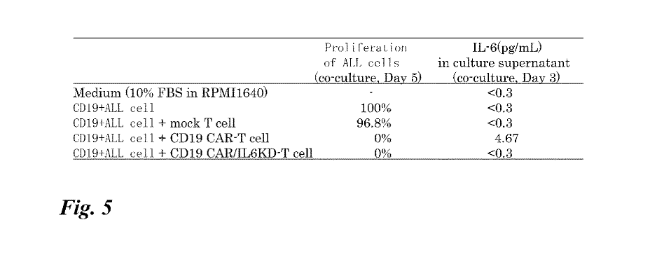

CD19 CAR expression vectors (pIRII-CAR.CD19-IL6KD and pIRII-CAR.CD19-TNFaKD) inhibiting the expression of the IL-6 gene or TNF-α gene were constructed (FIGS. 1 and 3). The T cells (CD19 CAR/IL6KD-T-cells) which expressed pIRII-CAR.CD19-IL6KD were used to study the inflammatory cytokine production inhibition and cytocidal effects of the CAR-T-cells. The results are shown in FIG. 5. The CD19 CAR-T-cells produce IL-6 by co-cultured with the CD19-positive ALL cells. However, the introduction of the shRNA sequence, which inhibits the expression of the IL-6 gene, into the CD19 CAR vector almost completely inhibited the IL-6 production from the CD19 CAR-T-cells. The introduction of the IL-6 shRNA sequence did not weaken the cytocidal effect of the CD19 CAR-T-cells.

3. Discussion

The CAR-T-cells after knockdown of gene expression of inflammatory cytokine exhibits antileukemic effect equivalent to the conventional CAR-T-cells, but does not release corresponding inflammatory cytokine. Enhancement of the effect can be expected by knocking down two or more genes of inflammatory cytokines simultaneously, or knocking down the upstream genes which promote the release of inflammatory cytokines.

The cytokine release syndrome has the problem of overexpression of IFN-γ, as well as TNF-α and IL-6. This suggests that inhibition of expression targeting IFN-γ gene is also an effective strategy.

### INDUSTRIAL APPLICABILITY

The present invention provides an effective measure for the cytokine release syndrome which is a problem in the CAR therapy. According to the present invention, the release of inflammatory cytokines from the CAR gene-introduced lymphocytes is inhibited. Accordingly, this measure is effective for prevention of development of serious complications, different from the prior art measure wherein therapeutic intervention is made after development of the cytokine release syndrome. More specifically, the use of the present invention allows more efficient, safe, and cost-effective prevention of the development of the cytokine release syndrome, which is the greatest fault of the CAR therapy, than the administration of the anti-IL-6 receptor antibody and anti-TNF-α antibody, and is expected to improve the treatment results. In particular, when the CAR therapy is used for the patient with tumor lesion in the central nervous system, there is a possibility of alleviation of central nervous system complications related to treatment.

The present invention is not limited only to the description of the above embodiments. A variety of modifications which are within the scopes of the following claims and which are achieved easily by a person skilled in the art are included in the present invention.

What is claimed is:

1. A method for preparing a gene-modified lymphocyte expressing chimeric antigen receptor, comprising a step of introducing a target antigen-specific chimeric antigen receptor gene and a first nucleic acid construct which intracellularly produces an siRNA targeting interleukin-6 gene, and/or a second nucleic acid construct which intracellularly produces an siRNA targeting tumor necrosis factor α gene into a target cell.

2. The preparation method of claim 1, wherein the introduction of the target antigen-specific chimeric antigen receptor gene, the first nucleic acid construct, and the second nucleic acid construct is carried out by a transposon method.

3. The preparation method of claim 2, wherein the transposon method is the piggyBac transposon method.

4. The preparation method of claim 1, wherein the target antigen-specific chimeric antigen receptor gene, the first nucleic acid construct and/or the second expression construct are included in the same vector, and the vector is introduced into the target cell.

5. The preparation method of claim 1, wherein the target cell is T-cell.

6. A gene-modified lymphocyte obtained by the preparation method of claim 1, which expresses the chimeric antigen receptor and intracellularly produces the siRNA targeting interleukin-6 gene and/or the siRNA targeting tumor necrosis factor α gene.

7. A method for treating cancer comprising a step of administering the gene-modified lymphocyte obtained by the preparation method of claim 1 to a cancer patient in a therapeutically effective amount, the gene-modified lymphocyte expressing chimeric antigen receptor, and intracellularly producing an siRNA targeting interleukin-6 gene and/or an siRNA targeting tumor necrosis factor α gene.


SEQUENCE LISTING (29 sequences)

SEQ ID NO: 1 - DNA, 1201 bp, Homo sapiens
aatattagagtctcaacccccaataaatataggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaaaaaaaaaaa

SEQ ID NO: 2 - DNA, 1686 bp, Homo sapiens
cagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaaaaaaaaaaaa

SEQ ID NO: 3 - RNA, 19 nt, Artificial Sequence
  IL-6 siRNA
gcacagaacuuauguuguu

SEQ ID NO: 4 - RNA, 59 nt, Artificial Sequence
  IL-6 shRNA
gcacagaacuuauguuguuuagugcuccugguugaacaacauaaguucugugcuuuuuu

SEQ ID NO: 5 - RNA, 19 nt, Artificial Sequence
  TNF-alpha siRNA
gucagaucaucuucucgaa

SEQ ID NO: 6 - RNA, 19 nt, Artificial Sequence
  TNF-alpha siRNA
gucuaaacaaugcugauuu

SEQ ID NO: 7 - RNA, 19 nt, Artificial Sequence
  TNF-alpha siRNA
gugaccaacugucacucau

SEQ ID NO: 8 - RNA, 62 nt, Artificial Sequence
  TNF-alpha shRNA
gucagaucaucuucucgaacacagggaagcgagucuguucgagaagaugaucugacuuuuuu

SEQ ID NO: 9 - RNA, 62 nt, Artificial Sequence
  TNF-alpha shRNA
gucuaaacaaugcugauuucacagggaagcgagucugaaaucagcauuguuuagacuuuuuu

SEQ ID NO: 10 - RNA, 62 nt, Artificial Sequence
  TNF-alpha shRNA
gugaccaacugucacucaucacagggaagcgagucugaugagugacaguuggucacuuuuuu

SEQ ID NO: 11 - DNA, 6680 bp, Artificial Sequence
  pIRII-CAR.CD19.IL6KD
tcgagctcaagcttcgaattcgaatggccatggagtttgggctgagctggctttttcttgtggctattttaaaaggtgtccagtgctctagagacatccagatgacacagactacatcctccctgtctgcctctctgggagacagagtcaccatcagttgcagggcaagtcaggacattagtaaatatttaaattggtatcagcaaaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagctgaaacgtggtggtggtggttctggtggtggtggttctggcggcggcggctccggtggtggtggatccgaggtgcagctgcagcagtctggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaagggaccacggtcaccgtctcctcgtacgtcaccgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaagcatgctagctatagttctagaggtaccggttgttaacgttagccggctacgtatactccggaatattaataggcctaggatgcatatggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccgcacagaacttatgttgtttagtgctcctggttgaacaacataagttctgtgcttttttaagcttgatatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgcccgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggattttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatctttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagataagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcc

SEQ ID NO: 12 - DNA, 6683 bp, Artificial Sequence
  pIRII-CAR.CD19.TNFaKD
tcgagctcaagcttcgaattcgaatggccatggagtttgggctgagctggctttttcttgtggctattttaaaaggtgtccagtgctctagagacatccagatgacacagactacatcctccctgtctgcctctctgggagacagagtcaccatcagttgcagggcaagtcaggacattagtaaatatttaaattggtatcagcaaaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagctgaaacgtggtggtggtggttctggtggtggtggttctggcggcggcggctccggtggtggtggatccgaggtgcagctgcagcagtctggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaagggaccacggtcaccgtctcctcgtacgtcaccgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaagcatgctagctatagttctagaggtaccggttgttaacgttagccggctacgtatactccggaatattaataggcctaggatgcatatggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccgtcagatcatcttctcgaacacagggaagcgagtctgttcgagaagatgatctgacttttttaagcttgatatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgcccgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggattttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatctttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagataagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcc

SEQ ID NO: 13 - DNA, 6683 bp, Artificial Sequence
  pIRII-CAR.CD19.TNFaKD
tcgagctcaagcttcgaattcgaatggccatggagtttgggctgagctggctttttcttgtggctattttaaaaggtgtccagtgctctagagacatccagatgacacagactacatcctccctgtctgcctctctgggagacagagtcaccatcagttgcagggcaagtcaggacattagtaaatatttaaattggtatcagcaaaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagctgaaacgtggtggtggtggttctggtggtggtggttctggcggcggcggctccggtggtggtggatccgaggtgcagctgcagcagtctggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaagggaccacggtcaccgtctcctcgtacgtcaccgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaagcatgctagctatagttctagaggtaccggttgttaacgttagccggctacgtatactccggaatattaataggcctaggatgcatatggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccgtctaaacaatgctgatttcacagggaagcgagtctgaaatcagcattgtttagacttttttaagcttgatatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgcccgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggattttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatctttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagataagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcc

SEQ ID NO: 14 - DNA, 6683 bp, Artificial Sequence
  pIRII-CAR.CD19.TNFaKD
tcgagctcaagcttcgaattcgaatggccatggagtttgggctgagctggctttttcttgtggctattttaaaaggtgtccagtgctctagagacatccagatgacacagactacatcctccctgtctgcctctctgggagacagagtcaccatcagttgcagggcaagtcaggacattagtaaatatttaaattggtatcagcaaaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagctgaaacgtggtggtggtggttctggtggtggtggttctggcggcggcggctccggtggtggtggatccgaggtgcagctgcagcagtctggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaagggaccacggtcaccgtctcctcgtacgtcaccgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaagcatgctagctatagttctagaggtaccggttgttaacgttagccggctacgtatactccggaatattaataggcctaggatgcatatggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccgtgaccaactgtcactcatcacagggaagcgagtctgatgagtgacagttggtcacttttttaagcttgatatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgcccgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggattttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatctttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagataagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcc

SEQ ID NO: 15 - DNA, 344 bp, Artificial Sequence
  DNA fragment for RNAi
caattgaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccgcacagaacttatgttgtttagtgctcctggttgaacaacataagttctgtgcttttttaagcttgatatcgat

SEQ ID NO: 16 - DNA, 258 bp, Artificial Sequence
  U6 promoter
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacga

SEQ ID NO: 17 - DNA, 59 bp, Artificial Sequence
  DNA coding IL6 shRNA
gcacagaacttatgttgtttagtgctcctggttgaacaacataagttctgtgctttttt

SEQ ID NO: 18 - DNA, 347 bp, Artificial Sequence
  DNA fragment for RNAi
caattgaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccgtcagatcatcttctcgaacacagggaagcgagtctgttcgagaagatgatctgacttttttaagcttgatatcgat

SEQ ID NO: 19 - DNA, 347 bp, Artificial Sequence
  DNA fragment for RNAi
caattgaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccgtctaaacaatgctgatttcacagggaagcgagtctgaaatcagcattgtttagacttttttaagcttgatatcgat

SEQ ID NO: 20 - DNA, 347 bp, Artificial Sequence
  DNA fragment for RNAi
caattgaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccgtgaccaactgtcactcatcacagggaagcgagtctgatgagtgacagttggtcacttttttaagcttgatatcgat

SEQ ID NO: 21 - DNA, 62 bp, Artificial Sequence
  DNA coding TNF-alpha shRNA
gtcagatcatcttctcgaacacagggaagcgagtctgttcgagaagatgatctgactttttt

SEQ ID NO: 22 - DNA, 62 bp, Artificial Sequence
  DNA coding TNF-alpha shRNA
gtctaaacaatgctgatttcacagggaagcgagtctgaaatcagcattgtttagactttttt

SEQ ID NO: 23 - DNA, 62 bp, Artificial Sequence
  DNA coding TNF-alpha shRNA
gtgaccaactgtcactcatcacagggaagcgagtctgatgagtgacagttggtcactttttt

SEQ ID NO: 24 - protein, 19 aa, Artificial Sequence
  Leader
MEFGLSWLFLVAILKGVQC

SEQ ID NO: 25 - protein, 110 aa, Artificial Sequence
  VL
SRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKR

SEQ ID NO: 26 - protein, 116 aa, Artificial Sequence
  VH
LQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVS

SEQ ID NO: 27 - protein, 232 aa, Artificial Sequence
  CH2CH3
EPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 68 aa, Artificial Sequence
  CD28
FWVLVVVGGVLACYSLLVTVAFIIFWVRSKRSRLLHSDYMNMTPRRPGPTRKHYQPYAPPRDFAAYRS

SEQ ID NO: 29 - protein, 112 aa, Artificial Sequence
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR